(19) United States
(12) Patent Application Publication
(10) Pub. No.: US 2007/0014848 A1
(43) Pub. Date: Jan. 18, 2007

(54) Resorbable Polyetheresters and Medicinal Implants Made Therefrom

(75) Inventors: Berthold Buchholz, Ockenheim (DE); Anja Enderle, Muehltal (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/457,190

(22) Filed: Jul. 13, 2006

(30) Foreign Application Priority Data
Jul. 15, 2005 (DE) 102005033101

(51) Int. Cl. A61K 9/64; C08F 20/02

(52) U.S. Cl. 424/456; 525/437

(57) ABSTRACT

The invention relates to the use of absorbable block copolymers with polyether and polyester units for preparing surgical implants which are suitable for the human or animal body, and the block copolymers in question. The block copolymers used according to the invention and those which are new according to the invention are characterised by high mechanical strength and rapid absorption kinetics.

The present invention relates to absorbable block copolymers with polyether and polyester units, hereinafter referred to as poly(etheresters), and the use thereof for preparing surgical or therapeutic implants for the human or animal body. The block copolymers according to the invention and implants prepared therefrom are characterised by improved absorption kinetics while simultaneously having high mechanical strength. In the course of the description the manufacture and purification of the block copolymers according to the invention will be discussed.

### PRIOR ART

Absorbable polymers are becoming increasingly important as additives in pharmaceutical formulations or in biodegradable implants. Polymers with distinctly different physical and chemical properties are required for all kinds of technical applications.

Thus, as additives for pharmaceutical formulations, block copolymers with polyester and polyether units are used inter alia as carrier materials for active substances or as materials for the microencapsulation of active substances. In this case the polymers are preferably administered by the parenteral route. In the case of carrier materials this method of use presupposes that the materials may be mixed with the other components of the formulation, either as powders, in solution or in suspension, without any loss of quality. In the case of microencapsulation it is also true that the polymer used behaves in a chemically and physically neutral manner with respect to the other ingredients of the formulation. A further requirement is that the microcapsule must release the active substance at the target site. Naturally brittle or firm materials are ruled out of such applications. In this field, poly(etheresters) of the AB type, ABA type, BAB type or ABABABAB type have proved suitable, inter alia. Examples include polymers with L- or D,L-lactide for the A-block and polyethyleneglycol for the B-block.

In addition to being used in the pharmaceutical field, absorbable polymers are increasingly attracting the interest of the specialists in surgery and surgical treatment. When choosing materials which appear to be suitable in this field, unlike in the pharmaceutical field, it is important to take into account the mechanical properties of the materials, as well as their toxicological properties. Solid materials are used which satisfy the toxicological and mechanical profile, on the one hand, but which also have properties which are essential for the manufacture and processing. Thus, the materials should be amenable to thermoplastic processing methods, such as injection moulding, molten pressing or extrusion or withstand the demands of mechanical methods such as machining. The essential properties in this respect are chiefly strength under tensile or torsional stress and speed of degradation.

The properties of an implant are determined primarily by the material used and less by its processing.

The absorbable implants have the advantage, over non-absorbable materials such as metals, that after they have fulfilled their function in the human or animal body they are broken down hydrolytically and the breakdown products are reabsorbed by the body. There is therefore no need to remove the implant in a second operation. A further advantage of the absorbable implants consists, for example, in the better toleration of the materials, as demonstrated by the example of osteosynthesis, where with non-absorbable implants there is the danger of atrophy of the bone by inactivity, which may lead to an increased risk of a fresh fracture of the bone once the implant is removed. The polymers which are of interest for surgical purposes, as well as being absorbable, should also have other properties such as high strength.

Known absorbable polymers which are suitable for surgical implants and the like include, for example, aliphatic poly(esters) based on lactide (=3,6-dimethyl-1,4-dioxan-e-dione), glycolide (=1,4-dioxane-2,5-dione), dioxanone (1,4-dioxan-2-one), copolymers of lactide/glycolide with trimethylene carbonate (=1,3-dioxan-2-one) and epsilon-caprolactone. Preferably, the ones with high molecular weights are used.

Examples include: poly(L-lactide), poly(D,L-lactide), poly(L-lactide-co-D,L-lactide), poly(D,L-lactide-co-glycolide), poly(glycolide), poly(L-lactide-co-glycolide), poly(glycolide-co-trimethylene carbonate), poly(L-lactide-co-trimethylene carbonate), poly(D,L-lactide-co-trimethylene carbonate), poly(L-lactide-co-caprolactone).

Using the above-mentioned materials it is possible to produce absorbable implants which cover a wide spectrum in terms of their mechanical properties. For example, poly(L-lactide) in addition to having high strength also has great rigidity and brittleness, whereas the copolymerisation of D,L-lactide and trimethylene carbonate results in materials with viscoplastic properties. In view of their breakdown rate of a few months to several years, these materials are particularly suitable for implants which are intended to remain in the body for a correspondingly long time.

On the other hand, there is often a hitherto unmet need in surgery for more rapidly degradable materials and implants. Particularly for paediatric applications and for implants for fixing fast-proliferating tissue, materials are needed which break down comparatively quickly but still have the necessary mechanical properties such as strength, elasticity, tenacity etc.

The present invention makes a contribution to the provision of such materials. In fact, it has been found that, surprisingly, by the ring-opening polymerisation of cyclic esters in the presence of mono- or difunctional poly(ethyleneglycol) under moderate synthesis conditions, high-molecular block copolymers of the AB or ABA type which are suitable for the manufacture of implants with improved properties can be produced simply on an industrially applicable scale. These polymers can also be purified on an industrial scale by simple methods, e.g. by extraction processes, so that their degree of purity meets the requirements for implantation into the body. In addition, they have properties which make it possible to process the materials by simple thermoplastic shaping methods to produce implants.

### AIM OF THE INVENTION

The aim of the present invention is to provide materials for preparing medical and surgical implants which are broken down more quickly by the human or animal body than materials known from the prior art, but at the same time have high mechanical strength, such as for example tensile strength.

A further aim is to provide materials for preparing medical and surgical implants which have the physical properties that are suitable for implants. These include for example initial strength, elasticity or tenacity.

A further aim of the invention is to provide the materials according to the invention with a degree of purity which allows them to be used in the human or animal body. Particular importance is attached to a low content of synthesis starting materials in the finished material.

A further aim of the invention is to provide materials for surgical implants which are absorbed rapidly enough to be used in paediatric applications or as implants for fixing fast-proliferating tissue.

A further aim of the invention is to provide a process which can be used on an industrial scale for preparing raw materials for the above-mentioned implants.

A further aim of the invention is to provide medical implants and processes for the preparation thereof.

### DESCRIPTION OF THE INVENTION

The invention relates to block copolymers consisting of polyester units and polyether units for preparing absorbable, surgical implants.

In a preferred aspect the present invention relates to implants containing a block copolymer according to the invention.

By the term implant is meant an (absorbable) moulded body which is suitable both surgically and mechanically for introduction into the human or animal body and is toxicologically unobjectionable. Such moulded bodies may be: screws, pins, plates, nuts for screws, anchors, fleeces, films, membranes, meshes, etc. They may be used to fix hard tissue fractures, as suture material anchors, as spinal implants, for closing and attaching blood and other vessels, as stents, for filling cavities or holes in tissue defects, etc.

The block copolymers according to the invention are also suitable for example for the preparation of drug eluting stents. These are vascular supports for placing in arterial vessels, which release proliferation-inhibiting active substances over a long period into the surrounding tissue to prevent restenosis. Active substances selected from among the cytostatics, such as paclitaxel, for example, have proved satisfactory for this purpose.

The implants according to the invention may be produced from the materials by thermoplastic forming methods to produce the shapes required for medical use, such as screws, for example. Suitable shaping processes are those known per se from the prior art for thermoplastics, such as molten pressing and preferably extrusion and injection moulding processes. Forming by injection moulding processes is particularly preferred.

The temperatures that are suitable for injection moulding depend on the precise copolymer composition and are in the range from 110 to 210° C. Higher processing temperatures are needed for block copolymers with a high molecular weight and hence a high melt viscosity than for polymers with a comparatively low molecular weight. Because of their crystallinity high processing temperatures are also needed for block copolymers with a high proportion of L-lactide units.

Because they are prone to hydrolytic decomposition it is also advantageous to dry the block copolymers before they are processed and to keep the processing temperatures as low as possible.

The block copolymers according to the invention used are polyetheresters of the AB or ABA type.

A denotes a polymer block with recurring ester units and B denotes a polymer block with recurring ether units.

The polyester block A consists of n components which can be traced back formally to one or more hydroxycarboxylic acids or a carbonate, preferably to an alpha-hydroxycarboxylic acid. If desired a block A may also formally be synthesised from several different ones of these components.

The letter B denotes a polyether unit, the repeating units of which are formally derived from ethyleneglycol. The repeating units may be present m-fold.

The polymers according to the invention are produced by synthesising the polyester block or blocks A on a polyethyleneglycol block B with one or two free terminal OH groups. Accordingly, polyethyleneglycol blocks with two free terminal OH groups are used for polymers of the ABA type, while polyethyleneglycol blocks with only one free terminal OH group are used for polymers of the AB type.

The polyetheresters of type AB according to the invention can be represented by general formula I:
E-(O-D-CO—)ₙ—(O—CH₂—CH₂—)ₘ—O—F Formula I:

The structural unit E-(-O-D-CO—)ₙ— forms the block A.

The block A is linked to the block B via a covalent bond.

D may denote, for each of the n units independently of one another:
—(CH(CH₃)—), or
—(CH₂—)ₓ or
—(CH₂—O—CH₂—CH₂—) or
—(CH₂—CH₂—CH₂—O—),

x is 1, 2, 3, 4 or 5 and

E is H, methyl or ethyl;

n is an integer greater than 1.

The structural unit —(O—CH₂—CH₂—)ₘ—O—F forms the block B.

F is H, methyl or ethyl;

m is an integer greater than 1.

It should be expressly pointed out that AB block copolymers are structurally identical to BA block copolymers, and for this reason no differentiation has been made within the scope of this description.

ABA block copolymers are polymers of general formula II on the basis of the above definitions.
E(-O-D-CO)Dₙ—(O—CH₂—CH₂—)ₘ—O—(CO-D-O—)_{n′}-E Formula II:

with all the variables in the definition provided above and n′ is an integer greater than 1.

As is normal with polymers, the letters n, n′ and m relate to numbers which describe the statistically average chain length of the two blocks. The precise figures in each individual molecule are subject to a statistical distribution.

The length of the B block in the copolymers may be between 500 and 10000 Dalton on average. An average block length of between 600 and 8000 Dalton is preferred, while an average block length of between 1000 and 8000 Dalton is particularly preferred. A short poly(ethyleneglycol) fragment which is released by the hydrolytic breakdown of the block copolymers can easily be excreted by the body through the kidneys.

At this point it should be mentioned that all the ranges specified in this description are inclusive in each case.

The weight ratio of the A block to the B block plays an essential role regarding the properties of the copolymers and the implants prepared therefrom according to the invention. The greater the amount of B block, the more hydrophilic the material, which has positive effects in terms of rapid absorbability (hydrolysis or breakdown rate).

According to the invention the proportion by weight of the B block is between 0.01 and 25%. The following sequence gives the preferred proportion of B in ascending order of priority for the individual embodiments: 0.01 to 20 wt. %, 0.1 to 15 wt. %, 0.1 to 10 wt. %, 0.1 to 5 wt. %, 0.1 to 4 wt. %, 1 to 3 wt. %. Particularly preferred are polymers with the last three amounts of B. In fact, it has surprisingly been found that, in these embodiments with only small amounts of the hydrophilic B block, the hydrolytic breakdown is speeded up considerably compared with the corresponding pure poly(esters) A.

Most preferred are polymers containing a proportion of B of 0.1 to 4 wt. %. This is particularly advantageous as mouldings which contain a block copolymer with a high dominance in the A block are stronger. Therefore, embodiments containing an amount of B of 1 to 3 wt. % and particularly 0.5 to 1.5 wt. % are even more preferred in this respect.

The above-mentioned details of the length of the block B and its proportion by weight in the polymer as a whole automatically give the block lengths of the A block or blocks and hence the total molecular weight. With triblock copolymers of type ABA it is to be assumed that the length and the proportion by weight of the two blocks A are equal, on average. Similarly, the total molecular weight of the copolymer is determined by the molecular weight of the ethyleneglycol used in the synthesis and the ratio of the two components put in.

It should be pointed out here that naturally the molecular weight of the final block copolymer may also be definitively determined by means of the length and proportion of block B.

The inherent viscosity (i.v.) may be used as another important parameter for characterising the polymers suitable for use according to the invention. The inherent viscosity of the block copolymers may vary over a wide range. An inherent viscosity (measured in an Ubbelohde viscosimeter in chloroform at 25° C. in 0.1 percent solution) of between 0.1 and 6 dl/g, preferably between 0.5 and 5 dl/g, is preferred. Particularly preferred are values of between 0.6 and 3 dl/g, and values of between 0.7 and 2.75 dl/g are most particularly preferred.

Block copolymers of the AB or ABA type having the following features are preferred according to the invention:
The inherent viscosity (measured in chloroform at 25° C. in 0.1 percent solution) is between 0.1 and 5.5 dl/g, preferably between 0.5 and 5.0 dl/g.
The average block length in the B block is between 1000 and 8000 Dalton.
The proportion by weight of the B block is between 0. 1 and 15 wt. %, preferably between 0.1 and 5 wt. %, more preferably between 0.1 and 4 wt. %.
Block A preferably has the following ester components:
   exclusively L-lactide, D-lactide, meso-lactide or DL-lactide,
   exclusively L-lactide,
   exclusively D,L-lactide,
   mixtures of statistically distributed (randomised) L-lactide and D,L-lactide, preferably with a molar proportion of L-lactide of between 60 and 90%,
   mixtures of statistically distributed (randomised) L-lactide and glycolide, preferably with a molar proportion of L-lactide of between 70 and 99%, more preferably between 85 and 99%, more preferably between 70 and 95%, more preferably between 87 and 95%.
   mixtures of statistically distributed (randomised) D,L-lactide and glycolide, preferably with a molar proportion of D,L-lactide of between 50 and 80%.
   mixtures of statistically distributed (randomised) L-lactide, D-lactide, meso-lactide or DL-lactide and epsilon-caprolactone units.
   mixtures of statistically distributed (randomised) L-lactide or D,L-lactide and epsilon-caprolactone units.
   mixtures of statistically distributed (randomised) L-lactide, D-lactide, meso-lactide or DL-lactide and dioxanone units.
   mixtures of statistically distributed (randomised) L-lactide or D,L-lactide and dioxanone units.
   mixtures of statistically distributed (randomised) L-lactide, D-lactide, meso-lactide or DL-lactide and trimethylene carbonate units.
   mixtures of statistically distributed (randomised) L-lactide or D,L-lactide and trimethylene carbonate units.

More preferred are block copolymers, wherein the block A preferably comprises the following ester components:
exclusively L-lactide,
exclusively D,L-lactide,
mixtures of statistically distributed (randomised) L-lactide and D,L-lactide, preferably with a molar proportion of L-lactide of between 60 and 90%,
mixtures of statistically distributed (randomised) L-lactide and glycolide, preferably with a molar proportion of L-lactide of between 70 and 95%.
mixtures of statistically distributed (randomised) D,L-lactide and glycolide, preferably with a molar proportion of D,L-lactide of between 50 and 80%.

The implants according to the invention preferably contain one or more different ones of the block copolymers according to the invention and no other additives besides, apart from impurities from the polymerisation process. In one particular embodiment the implants may also contain blends of high-molecular block copolymers with other absorbable materials, such as for example absorbable poly(esters) selected from among the poly(L-lactide), poly(D-lactide), poly(D,L-lactide), poly(meso-lactide), poly(glycolide), poly(trimethylene carbonate), poly(dioxanone), poly(epsilon-caprolactone), as well as copolymers of the corresponding heterocyclic groups or polyethyleneglycol. Blends in which the chemical structure of the A block in the block copolymer corresponds to that in the poly(ester) are preferred. This ensures good phase coupling in the blend, which is advantageous in terms of achieving good mechanical properties. It is also possible to use blends of different block copolymers.

The implants according to the invention may have a weight of between 1 and 10000 mg, preferably between 5 and 5000 mg and particularly preferably a weight of between 10 and 1000 mg.

The preferred tensile strength of the implants measured as mouldings according to the ASTM Standard D 638, which are produced according to the invention from the polymers described above, is at least 70 MPa, preferably 75 to 95 MPa, particularly preferably 80 to 88 MPa.

The preferred rate of hydrolysis (breakdown rate) of the implants, determined by means of the changes in the inherent viscosity on mouldings according to ASTM Standard D 638, which are produced according to the invention from the polymers described above, is at least 30%, preferably 40%, particularly preferably 45% based on the starting value, in a bath consisting of an aqueous phosphate buffer at pH 7.4 at a temperature of 37° C. after 6 weeks.

The invention further relates to an implant, characterised in that it contains a blend consisting of:
(a) a block copolymer according to the invention and
(b) an absorbable polyester A with recurring units of a lactide, selected from among L-lactide, D-lactide, DL-lactide or meso-lactide, glycolide, trimethylene carbonate, epsilon-caprolactone or dioxanone, wherein only similar or different ones of the specified units may be present.

Preparation of the Block Copolymers

Copolymers of the AB type may be synthesised by ring-opening polymerisation of cyclic esters in the presence of poly(ethyleneglycol) with a free hydroxyl group and a non-reactive end group, methoxy end groups being preferred. Copolymers of the ABA type may be produced in the presence of poly(ethyleneglycol) with two free hydroxyl groups. In this case the two blocks A are synthesised in parallel in the same synthesis step.

Cyclic esters of general formula III serve as components for the ring-opening polymerisation for preparing the block A or the blocks A.

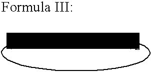

wherein D in each of the units -O-D-CO may independently of one another have one of the above-mentioned definitions.

z is a whole number and is at least 1, preferably 1 or 2. Particularly preferably, dimeric cyclic esters of alpha-hydroxycarboxylic acids, monomeric lactones or cyclic carbonates are used.

When producing the block or blocks A it is not essential to use only one type of component according to formula III. It is also possible to use mixtures which differ by the chemical nature of the structural element D.

Preferred structures according to formula III, or preferred molecules from which the blocks A are formed by ring-opening polymerisation, are a) L-lactide, b) D-lactide, c) mixtures of D- and L-lactide, such as e.g. racemic D,L-lactide, d) meso-lactide, e) glycolide, f) trimethylene carbonate, g) epsilon-caprolactone, h) dioxanone, i) mixtures of L-lactide and D,L-lactide, j) mixtures of L-lactide and glycolide, k) mixtures of D,L-lactide and glycolide, l) mixtures of L-lactide or D,L-lactide and trimethylene carbonate, m) mixtures of L-lactide or D,L-lactide and epsilon-caprolactone.

Particularly preferred are a) L-lactide, b) L-lactide and glycolide, c) L-lactide and D,L-lactide, d) D,L-lactide and glycolide in the ratios specified above (cf. the description of the polymers).

For preparing the poly(etheresters) it is advisable to use the raw materials in a high degree of purity. Particularly polar-protic impurities such as e.g. water lead to chain breakage during polymerisation. For this reason it is advisable to dry the poly(ethyleneglycol) before use in the polymerisation.

For the synthesis poly(ethyleneglycol) is mixed with one or more of the monomers or dimers according to formula III and melted. After the educts have been homogeneously mixed, the catalyst intended for the ring-opening polymerisation is added. The reaction mixture is preferably polymerised at elevated temperature.

A number of different metal catalysts such as for example tin or zinc compounds are suitable for the synthesis. Tin(II)chloride or tin (II)ethylhexanoate is preferably used. In view of the proposed use in the human or animal body it is advantageous to use the smallest possible amount of catalyst. A concentration of between 30 and 200 ppm is preferred and a concentration of between 50 and 100 ppm is particularly preferred. The concentration given for the catalyst in each case refers to parts by weight of the catalysing metal ion, based on the total reaction mass.

The reaction temperature is above the melting temperature of the educts used in each case and therefore depends on the structure of the monomer(s) or dimer(s) according to formula III and the molecular weight of the poly(ethyleneglycol) used. Normally the work is done at a temperature range of between 100 and 160° C. A range of between 100 and 140° C. is preferred, while between 110 and 130° C. is particularly preferred. In polymers in which good solubility in organic solvents is important, the reaction temperature may be adjusted to 150° C. to 170° C. A higher reaction temperature favours a good statistical distribution of the comonomer units in the A block or in the A blocks. In this way it is possible to prepare, for example, glycolide-containing polymers which dissolve readily in acetone.

The necessary reaction times depend on the reaction speed of the monomer(s) or dimer(s) of formula III used, the reaction temperature and also the catalyst concentration and range between a few hours and several days. A reaction time of between 24 hours and 5 days is preferred, while a time of between 2 and 5 days is particularly preferred. Longer reaction times generally bring about a higher conversion, which in turn contributes to a reduction in the concentration of the educts in the end product.

To sum up, the relevant reaction parameters, namely the amount of catalyst, the reaction temperature and reaction time are selected, depending on the educts used, from the point of view of the lowest possible catalyst content, a moderate reaction temperature for avoiding reactions of decomposition and discoloration in the product and the most extensive possible reaction of the monomers or dimers.

As a rule the polymer prepared according to the above description is also subjected to a purification process. As the ring-opening polymerisation of the educts according to formula III is an equilibrium reaction, traces of unreacted educts are still present in the crude polymers, which may be detrimental to subsequent processing and implantation.

The purification of the polymers may be carried out by precipitation from various solvents or by extraction.

For the precipitation, the crude polymer is dissolved in a solvent which is miscible with precipitation agent, e.g. acetone, methylethylketone, glacial acetic acid, a mixture of glacial acetic acid and acetone, DMSO, methylene chloride, chloroform or a mixture of methylene chloride and chloroform, and the solution obtained is mixed with water, methanol, ethanol or other alcohols as precipitation agent. Other solvents/precipitation agents are conceivable (e.g. precipitation in ether), but are not preferred on an industrial scale on account of toxicities or safety considerations. Amorphous polymers, which are difficult or impossible to purify by extraction, are often purified by precipitation.

An extraction process is preferred. The crude polymer obtained is extracted with a solvent and then dried. For the extraction the crude polymers are usually ground up beforehand to ensure adequate diffusion of the solvent into the solid. For the purification process organic solvents and supercritical or pressure-liquefied gases are suitable, which dissolve the monomer but not the polymer. Preferably organic solvents selected from among the n-alkanes or cyclo-alkanes (cyclo-hexane or n-hexane at ambient temperature) and carbon dioxide are used, particularly preferably supercritical or pressure-liquefied carbon dioxide is used.

The present invention therefore further relates to a process for preparing the poly(etheresters) of the AB and ABA type according to the invention, comprising the steps of:
(a) mixing and melting poly(ethyleneglycol) with one or two free terminal OH groups together with one or more monomers or dimers according to formula III, 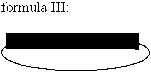
wherein D in each of the units -O-D-CO independently of one another may be:
   —(CH(CH₃)-—ₓ or
   —(CH₂—)ₓ or
   —(CH₂—O—CH₂—CH₂—) or
   —(CH₂—CH₂—CH₂—O—),
   x is 1, 2, 3, 4 or 5,
and z is an integer and at least 1, preferably 1 or 2,
(b) adding a metal catalyst to the homogeneous mixture of (a) obtained,
(c) polymerising the mixture of (b) at a reaction temperature which is above the melting temperature of the educts used, for a reaction time of between 24 hours and 5 days,
(d) purifying the crude polymer obtained in step (c) by precipitation from a solvent or by extraction and
(e) comminuting the polymer obtained in step (d).

Preferably the poly(ethyleneglycol) used in step (a) is dried beforehand. The more preferred embodiments may be inferred from the foregoing remarks.

Preparation of the Implants

The resulting high-molecular block copolymers can easily be processed by thermoplastic forming into surgical implants which have the desired faster breakdown characteristics, compared with the implants known from the prior art, while still having a high initial strength.

Use of the Implants According to the Invention

The implants according to the invention may be used for example for fixing hard tissue fractures (osteosynthesis), for controlled tissue regeneration in the soft tissues, for fixing suture threads in the bone (suture material anchor), as spinal implants for protecting the intervertebral ligaments (e.g. so-called “spinal cages”), for closing and attaching blood and other vessels in vessel ruptures (anastomosis), as stents, for filling cavities or holes in tissue defects, for example in dentistry or in defects of the septum of the heart and for fixing tendons and ligaments in the bone. For this, the block copolymers are processed into mouldings the design of which is adapted to the particular application. Thus, for example, screws, pins, plates, nuts, anchors, fleeces, films, membranes, meshes and the like may be obtained. Screws of this kind may for example be made in various sizes, with different thread sizes, with a right- or left-handed thread and with different screw heads, e.g. cross-heads or single slots. Other possible uses may be inferred from the prior art.

### EXAMPLES

The following Examples serve as exemplifying illustrations and are to be interpreted only as possibilities, without restricting the invention to their content.

1. In Vitro Degradation Study

The polymers according to the invention are processed using injection moulding processes known from the prior art to form mouldings (testpieces). These are fixed in a wire mesh and are thus placed in a hydrolysis bath regulated to a temperature of 37° C. This is filled with a phosphate buffer solution at pH 7.4, which is changed weekly. Samples are taken at fixed testing times. The breakdown of the polymers is observed through the change in the parameter of inherent viscosity (i.v.) over time (measured in an Ubbelohde viscosimeter in chloroform at 25° C. in 0.1 percent solution).

1.1 Poly L-lactide-polyethyleneglycol di- or triblock copolymers

The following are used:
polymers of the ABA type with L-lactide as component of the A block and 1 and 5% polyethyleneglycol 6000 (PEG 6000) as B block and
polymers of the AB type with L-lactide as component of the A block and 1 and 5% polyethyleneglycol 2000 (PEG 2000) as B block. The numbers 2000 and 6000 indicate the molar mass of the PEG in Daltons.

The value determined for the inherent viscosity at time 0 is standardised to 100%. This corresponds to the value before the testpiece has been dipped into the hydrolysis bath. To determine the breakdown, the measured values are recorded in % based on the starting value.

The following are used as AB polymers:

sample 1: L-lactide-polyethyleneglycol with a proportion by weight of polyethyleneglycol (PEG) of 1% and a molar mass of 2000 Dalton. The length of the A block is thus calculated as 198000 Dalton.

sample 2: L-lactide-polyethyleneglycol with a proportion by weight of polyethyleneglycol (PEG) of 5% and a molar mass of 2000 Dalton. The length of the A block is thus calculated as 38000 Dalton.

The following are used as ABA polymers:

sample 3: L-lactide-polyethyleneglycol-L-lactide with a proportion by weight of polyethyleneglycol (PEG) of 1% and a molar mass of 6000 Dalton. The length of the A blocks is thus calculated as 297000 Dalton in each case.

sample 4: L-lactide-polyethyleneglycol-L-lactide with a proportion by weight of polyethyleneglycol (PEG) of 5% and a molar mass of 6000 Dalton. The length of the A blocks is thus calculated as 57000 Dalton in each case.

Result:

Table 1 that follows gives an overview of the breakdown rates of the polymers used:

| | poly | | | | | | | | | |
|---|---|---|---|---|---|---|---|---|---|---|
| | L-lactide | | | | | | | | | |
| | (comparison) | | sample 1 | | sample 2 | | sample 3 | | sample 4 | |
| | i.v. | | i.v. | | i.v. | | i.v. | | i.v. | |
| duration [weeks] | [dl/g] | % | [dl/g] | % | [dl/g] | % | [dl/g] | % | [dl/g] | % |
| 0 | 2.64 | 100 | 1.96 | 100 | 1.12 | 100 | 2.49 | 100 | 1.55 | 100 |
| 2 | 2.45 | 93 | 1.88 | 96 | 1.07 | 96 | 2.02 | 81 | 1.31 | 85 |
| 4 | n.d. | n.d. | 1.78 | 91 | 0.96 | 86 | 1.57 | 63 | 0.99 | 64 |
| 5 | 2.27 | 86 | n.d. | n.d. | n.d. | n.d. | n.d. | n.d. | n.d. | n.d. |
| 6 | n.d. | n.d. | 1.70 | 87 | 0.84 | 75 | 1.25 | 50 | 0.79 | 51 |
| 8 | n.d. | n.d. | 1.50 | 77 | 0.73 | 65 | 1.05 | 42 | 0.66 | 43 |
| 10 | n.d. | n.d. | 1.41 | 72 | 0.67 | 60 | 0.95 | 38 | 0.58 | 37 |
| 11 | 1.92 | 73 | n.d. | n.d. | n.d. | n.d. | n.d. | n.d. | n.d. | n.d. |
| 12 | n.d. | n.d. | 1.28 | 65 | 0.59 | 53 | 0.80 | 32 | 0.50 | 32 |
| 14 | n.d. | n.d. | 1.22 | 62 | 0.54 | 48 | 0.72 | 29 | 0.48 | 31 |
| 15 | 1.70 | 65 | n.d. | n.d. | n.d. | n.d. | n.d. | n.d. | n.d. | n.d. |
| 16 | n.d. | n.d. | 1.10 | 56 | 0.45 | 40 | 0.61 | 24 | 0.38 | 25 |
| 18 | n.d. | n.d. | 1.02 | 52 | 0.42 | 38 | 0.56 | 22 | 0.36 | 23 |
| 20 | 1.57 | 59 | n.d. | n.d. | n.d. | n.d. | n.d. | n.d. | n.d. | n.d. |

i.v. inherent viscosity (measured in an Ubbelohde viscosimeter in chloroform at 25° C. in 0.1 percent solution)
n.d. not determined

In the AB diblock copolymers the following values are obtained after 18 weeks:

sample 1: 52% of the starting value

sample 2: 38% of the starting value

In the ABA triblock copolymers (samples 3 and 4) the hydrolytic breakdown after 18 weeks, regardless of whether 1 or 5% PEG had been chosen, leads to an i.v. which constitutes only about 25% of the starting value.

As a comparison: the value for poly L-lactide is in the region of barely 60% after 20 weeks.

1.2 Poly L-lactide-co-D,L-lactide-polyethyleneglycol di- or triblock copolymers

The following are used:
polymer of the ABA type with L-lactide-co-D,L-lactide as component of the A block and 5% polyethyleneglycol 2000 (PEG 2000) as B block and
polymers of the AB type with L-lactide-co-D,L-lactide as component of the A block and 5% polyethyleneglycol-MME 5000 (PEG-MME 5000) as B block. The numbers 2000 and 5000 respectively indicate the molar mass of the PEG or PEG-MME in Daltons.

The value determined for the inherent viscosity at time 0 is standardised to 100%. This corresponds to the value before the testpiece has been dipped into the hydrolysis bath. To determine the breakdown, the measured values are recorded in % based on the starting value.

The following are used as ABA polymers:

sample 5: L-lactide-co-D,L-lactide -polyethyleneglycol-L-lactide-co-D,L-lactide with a proportion by weight of polyethyleneglycol (PEG) of 5% and a molar mass of 2000 Dalton. The length of the A blocks is thus calculated as 19000 Dalton in each case.

The following are used as AB polymers:

sample 6: L-lactide-co-D,L-lactide -polyethyleneglycol with a proportion by weight of polyethyleneglycol (PEG) of 5% and a molar mass of 5000 Dalton. The length of the A block is thus calculated as 95000 Dalton.

Result:

In the ABA triblock copolymer the hydrolytic breakdown leads after 18 weeks to an i.v. which constitutes only about 30% of the starting value.

In the AB diblock copolymer (sample 6) after 18 weeks a viscosity value of 9% of the starting value is obtained.

1.3 Poly L-lactide-co-glycolide-polyethyleneglycol di- or triblock copolymers

The following are used:
polymer of the ABA type with L-lactide-co-glycolide as component of the A block and 5% polyethyleneglycol 2000 (PEG 2000) as B block and
polymers of the AB type with L-lactide-co-glycolide as component of the A block and 5% polyethyleneglycol-MME 5000 (PEG-MME 5000) as B block. The numbers 2000 and 5000 indicate the molar mass of the PEG and PEG-MME, respectively, in Daltons.

The value determined for the inherent viscosity at time 0 is standardised to 100%. This corresponds to the value before the testpiece has been dipped into the hydrolysis bath. To determine the breakdown, the measured values are recorded in % based on the starting value.

The following are used as ABA polymers:

sample 7: L-lactide-co-glycolide-polyethyleneglycol-L-lactide-co-glycolide with a proportion by weight of polyethyleneglycol (PEG) of 5% and a molar mass of 2000 Dalton. The length of the A blocks is thus calculated as 19000 Dalton in each case.

The following are used as AB polymers:

sample 8: L-lactide-co-glycolide-polyethyleneglycol with a proportion by weight of polyethyleneglycol (PEG) of 5% and a molar mass of 5000 Dalton. The length of the A block is thus calculated as 95000 Dalton.

Result:

Table 2 that follows provides an overview of the breakdown rates of the polymers used:

| | sample 5 | | sample 6 | | sample 7 | | sample 8 | |
|---|---|---|---|---|---|---|---|---|
| duration | i.v. | | i.v. | | i.v. | | i.v. | |
| [weeks] | [dl/g] | % | [dl/g] | % | [dl/g] | % | [dl/g] | % |
| 0 | 0.86 | 100 | 1.49 | 100 | 0.89 | 100 | 1.24 | 100 |
| 2 | 0.83 | 97 | 1.35 | 91 | 0.81 | 91 | 0.93 | 75 |
| 4 | 0.79 | 92 | 1.12 | 75 | 0.67 | 75 | 0.62 | 50 |
| 6 | 0.72 | 84 | 0.85 | 57 | 0.50 | 56 | 0.46 | 37 |
| 8 | 0.62 | 72 | 0.55 | 37 | 0.38 | 43 | 0.29 | 23 |
| 10 | 0.52 | 60 | 0.42 | 28 | 0.25 | 28 | 0.20 | 16 |
| 12 | 0.41 | 48 | 0.29 | 19 | 0.18 | 20 | 0.17 | 14 |
| 14 | 0.34 | 40 | 0.21 | 14 | 0.14 | 16 | 0.13 | 10 |
| 16 | 0.28 | 33 | 0.18 | 12 | 0.11 | 12 | 0.10 | 8 |
| 18 | 0.25 | 29 | 0.14 | 9 | 0.10 | 11 | 0.13 | 10 |

i.v. inherent viscosity (measured in an Ubbelohde viscosimeter in chloroform at 25° C. in 0.1 percent solution)

In both the ABA triblock copolymer and the AB diblock copolymer the hydrolytic breakdown leads after 18 weeks to an inherent viscosity which constitutes only about 10% of the starting value.

2. Mechanical Tests

To determine the tensile strength the testpieces listed below are produced according to ASTM D 638 and subjected to measurements:

sample 1: L-lactide-polyethyleneglycol with a proportion by weight of polyethyleneglycol (PEG) of 1% and a molar mass of 2000 Dalton. The length of the A block is thus calculated as 198000 Dalton.

sample 2: L-lactide-polyethyleneglycol with a proportion by weight of polyethyleneglycol (PEG) of 5% and a molar mass of 2000 Dalton. The length of the A block is thus calculated as 38000 Dalton.

sample 3: L-lactide-polyethyleneglycol-L-lactide with a proportion by weight of polyethyleneglycol (PEG) of I% and a molar mass of 6000 Dalton. The length of the A blocks is thus calculated as 297000 Dalton in each case.

sample 4: L-lactide-polyethyleneglycol-L-lactide with a proportion by weight of polyethyleneglycol (PEG) of 5% and a molar mass of 6000 Dalton. The length of the A blocks is thus calculated as 57000 Dalton in each case.

sample 5: L-lactide-polyethyleneglycol-L-lactide with a proportion by weight of polyethyleneglycol (PEG) of 15% and a molar mass of 6000 Dalton.

sample 6: L-lactide-co-D,L-lactide-polyethyleneglycol-L-lactide-co-D,L-lactide with a proportion by weight of polyethyleneglycol (PEG) of 5% and a molar mass of 2000 Dalton and a ratio of L-lactide to D,L-lactide =70:30.

sample 7: L-lactide-co-D,L-lactide-polyethyleneglycol with a proportion by weight of polyethyleneglycol (PEG) of 5% and a molar mass of 5000 Dalton and a ratio of L-lactide to D,L-lactide =70:30.

sample 8: L-lactide-co-glycolide-polyethyleneglycol-L-lactide-co-glycolide with a proportion by weight of polyethyleneglycol (PEG) of 5% and a molar mass of 2000 Dalton and a ratio of L-lactide to glycolide =85:15.

sample 9: L-lactide-co-glycolide-polyethyleneglycol with a proportion by weight of polyethyleneglycol (PEG) of 5% and a molar mass of 5000 Dalton and a ratio of L-lactide to glycolide =85:15.

sample 10: poly(L-lactide-co-glycolide) with a molar ratio of L-lactide to glycolide of 85:15*

sample 11: poly(L-lactide-co-DL-lactide) with a molar ratio of L-lactide to DL-lactide of 70:30*

The tensile strength of these reference materials was determined according to DIN 53455, the corresponding testpieces were produced according to DIN 53452.

Table 3 that follows provides an overview of the values obtained for the tensile strength of the testpieces used:

| | | tensile strength |
|---|---|---|
| Name | i.v. [dl/g] | [MPa] |
| sample 1 | 1.96 | 84 |
| sample 2 | 1.12 | 68 |
| sample 3 | 2.49 | 85 |
| sample 4 | 1.55 | 73 |
| sample 5 | 0.6 | 23 |
| sample 6 | 0.86 | 49 |
| sample 7 | 1.5 | 55 |
| sample 8 | 0.89 | 52 |
| sample 9 | 1.7 | 65 |
| sample 10 | 2.99 | 84 |
| sample 11 | 3.06 | 74 |

i.v. inherent viscosity (measured in an Ubbelohde viscosimeter in chloroform at 25° C. in 0.1 percent solution)

3. Polymers

3.1 Polylactide-polyethyleneglycol-polylactide triblock copolymer

35 g PEG 6000 (polyethyleneglycol with a molecular weight of 6000 Dalton, two terminal OH groups) are dried at 85° C. and 50 mbar/2 hours. 3.5 kg of L-lactide are added. At 112° C., 965 mg of tin(II)-2-ethylhexanoate is added to the molten educts. The mixture is bulk-polymerised for 3 days at 120° C. The resulting crude polymer is ground up and extracted under the conditions specified below. The polymer has an i.v. of 2.63 dl/g and a residual monomer content of lactide of less than 0.5%. The content of PEG in the copolymer is 0.9% (¹H-NMR). This corresponds to a molar mass for the A block of 338 000 g/mol per A block, based on the PEG 6000. The glass transition temperature (Tg) (by DSC measurement, DSC 200 PC made by Messrs Netsch, heating rate: 10° K/min) is 60.8° C.

3.2 Poly-D,L-lactide-co-L-lactide-polyethyleneglycol-poly-DL-lactide-co-L-lactide-triblock copolymer

175 g PEG 2000 (polyethyleneglycol with a molecular weight of 2000 Dalton, two terminal OH groups) are dried at 85° C. and 50 mbar/2 hours. 2520 g of L-lactide and 980 g of D,L-lactide are added. At 1 12° C., 1003 mg tin(I)-2-ethylhexanoate is added to the molten educts. The mixture is bulk-polymerised at 120° C. for 3 days. The resulting crude polymer is ground up and extracted under the conditions specified below. The polymer has an i.v. of 0.86 dl/g and a residual monomer content of lactide of less than 0.5%. The content of PEG in the copolymer is 4.8% (¹H-NMR).

3.3 Poly-L-lactide-co-glycolide-polyethyleneglycol diblock copolymer

125 g PEG-MME 2000 (polyethyleneglycol with a molecular weight of 2000 Dalton, a terminal OH group and a terminal methoxy group) are dried at 85° C. and 50 mbar/2 hours. 2135.2 g of L-lactide and 364.8 g of glycolide are added. At 112° C., 717 mg tin(II)-2-ethylhexanoate is added to the molten educts. The mixture is bulk-polymerised at 150° C. for 3 days. The resulting crude polymer is ground up and extracted. The polymer has an i.v. of 1.7 dl/g and a residual monomer content of less than 0.5%. The content of PEG in the copolymer is 5.3% (¹H-NMR).

3.4 poly-D,L-lactide-co-glycolide-polyethyleneglycol diblock copolymer

236.7 g PEG-MME 5000 (polyethyleneglycol with a molecular weight of 5000 Dalton, a terminal OH group and a terminal methoxy group) are dried at 85° C. and 50 mbar/2 hours. 2537 g of D,L-lactide and 1936 g of glycolide are added. At 112° C. 1293 mg tin(II)-2-ethylhexanoate is added to the molten educts. The mixture is bulk-polymerised at 150° C. for 3 days. The resulting crude polymer is purified by dissolving in acetone and precipitating in water and then dried. The polymer has an i.v. of 1.1 dl/g and a residual monomer content of less than 0.5%. The content of PEG in the copolymer is 4.9% (¹H-NMR).

3.5 poly-L-lactide-polyethyleneglycol-MME diblock copolymer

35 g PEG-MME 2000 (polyethyleneglycol with a molecular weight of 2000 Dalton, a terminal OH group and a terminal methoxy group) are dried at 85° C. and 50 mbar/2 hours. 3500 g of L-lactide are added. At 112° C., 965 mg of tin(II)-2-ethylhexanoate is added to the molten educts. The mixture is bulk-polymerised at 120° C. for 5-7 days. The resulting crude polymer is ground up and extracted. The polymer has an i.v. of 1.91 dl/g and a residual monomer content of less than 0.5%. The content of PEG in the copolymer is 0.94% (¹H-NMR).

3.6 Poly-L-lactide-co-D,L-lactide-polyethyleneglycol-MME diblock copolymer

175 g PEG-MME 5000 (polyethyleneglycol with a molecular weight of 5000 Dalton, a terminal OH group and a terminal methoxy group) are dried at 85° C. and 50 mbar/2 hours. 2520.0 g of L-lactide and 980 g of D,L-lactide are added. At 112° C., 1003 mg tin(II) 2-ethylhexanoate is added to the molten educts. The mixture is polymerised at 120° C. for 3 days. The resulting crude polymer is ground up and extracted. The polymer has an i.v. of 1.55 dl/g and a residual monomer content of less than 0.5%. The content of PEG in the copolymer is 4.84% (¹H-NMR).

3.7 Poly-L-lactide-co-glycolide-polyethyleneglycol-poly-L-lactide-co-glycolide triblock copolymer

65 g PEG-6000 (polyethyleneglycol with a molecular weight of 6000 Dalton, two terminal OH groups) are dried at 85° C. and 50 mbar/2 hours. 5496.1 g of L-lactide and 938.9 g of glycolide are added. At 112° C., 1775 mg of tin(II) 2-ethylhexanoate is added to the molten educts. The mixture is bulk-polymerised at 150° C. for 3 days. The resulting crude polymer is ground up and extracted. The polymer has an i.v. of 2.7 dl/g and a residual monomer content of less than 0.5%. The content of PEG in the copolymer is 1.0% (¹H-NMR).

3.8 Poly-D,L-lactide-co-glycolide-polyethyleneglycol-poly-D,L-lactide-co-glycolide triblock copolymer

499.5 g PEG 6000 (polyethyleneglycol with a molecular weight of 6000 Dalton, two terminal OH groups) are dried at 85° C. and 50 mbar/2 hours. 2537.0 g of D,L-lactide and 1963.0 g glycolide are added. At 112° C., 1365 mg tin(II)-2-ethylhexanoate is added to the molten educts. The mixture is bulk-polymerised at 150° C. for 3 days. The resulting crude polymer is purified by dissolving in acetone and precipitating in water and then dried. The polymer has an i.v. of 0.75 dl/g and a residual monomer content of less than 0.5%. The content of PEG in the copolymer is 10.05% (¹H-NMR).

4. Purification

The ground-up products of Examples 3.1 to 3.3 and 3.5 to 3.7 are placed in a 16 L extraction cartridge. The cartridge is closed and the contents are then extracted with pressure-liquefied carbon dioxide.

Extraction Conditions:

| | |
|---|---|
| time/pressure: | 1 h at 90 bar, followed by 4 h at 300 bar |
| temperature: | ≦10° C. |
| flow of carbon dioxide: | approx. 120 kg/h |

This purification example can be carried out analogously for other polymers.

What is claimed is:

1. An implant comprising a poly(etherester) which is an AB or ABA block copolymer, wherein A is a polyester block and B is a polyether block.

2. An implant according to claim 1 wherein B is a polyethyleneglycol.

3. An implant according to claim 1, wherein the polyetherester is of the AB type and is represented by formula I:
E-(O-D-CO—)ₙ—(O—CH₂—CH₂—)ₘ—O—F (formula I)
wherein
the structural unit E-(-O-D-CO—)ₙ— forms the block A,
—(O—CH₂—CH₂—)ₘ— forms the block B,
D may denote, for each of the n units independently of one another:
—(CH(CH₃)—)ₓ or
—(CH₂—)ₓ or
—(CH₂—O—CH₂—CH₂—) or
—(CH₂—CH₂—CH₂—O—),
x is 1,2, 3,4 or 5,
E and F independently of one another denote H, methyl or ethyl and
n and m are statistically averaged and independently of one another denote an integer greater than 1.

4. An implant according to claim 1, wherein the polyetherester is of the ABA type and is represented by formula II:
E(-O-D-CO)ₙ—(O—CH₂—CH₂—)ₘ—O—(CO-D-O—)_{n′}-E (formula II),
wherein
the structural units E-(-O-D-CO—)ₙ— and E-(-O-D-CO—)_{n′}— form the blocks A,
—(O—CH₂—CH₂—)ₘ— forms the block B,
D may be, for each of the n or n′ units independently of one another:
—(CH(CH₃)—)ₓ or
—(CH₂—)ₓ or
—(CH₂—0-CH₂—CH₂—) or
—(CH₂—CH₂—CH₂—O—),
x is 1, 2, 3, 4 or 5,
E is H, methyl or ethyl and
n, n′ and m are statistically averaged and independently of one another denote an integer greater than 1.

5. An implant according to claim 1, wherein A is synthesised from monomer components selected from the group consisting of a) L-lactide, b) D-lactide, c) mixtures of D- and L-lactide, preferably in the ratio 1: 1, d) meso-lactide, e) glycolide, f) trimethylene carbonate, g) epsilon-caprolactone, h) dioxanone, i) mixtures of L- and D,L-lactide, j) mixtures of L-lactide and glycolide, k) mixtures of D,L-lactide and glycolide l) mixtures of L-lactide or D,L-lactide and trimethylene carbonate, m) mixtures of L-lactide or D,L-lactide and epsilon-caprolactone and n) mixtures of L-lactide or D,L-lactide and dioxanone.

6. An implant according to claim 5, wherein A is synthesised from monomer components selected from the group consisting of a) L-lactide, b) L-lactide and glycolide, c) L-lactide and D,L-lactide, d) D,L-lactide and glycolide.

7. An implant according to claim 1, wherein the chain length of the block B is on average between 500 and 10000 Dalton and the proportion by weight of the B block is between 0.01 and 25 wt. %.

8. An implant according to claim 1 as a stent, which further comprises a cytostatic substance as active ingredient.

9. An implant according to claim 1, having an initial tensile strength of at least 70 MPa, consisting of a material which has a breakdown rate, measured by the loss of inherent viscosity, of more than 30% of the starting value after 6 weeks' hydrolysis in an aqueous phosphate buffer solution with a pH of 7.4 at 37° C.

10. An implant according to claim 1, consisting of a material which is in the form of a moulding according to ASTM D 638, has an initial tensile strength of at least 70 MPa, and which has a breakdown rate measured by the loss of the inherent viscosity of more than 30% of the starting value after 6 weeks' hydrolysis in an aqueous phosphate buffer solution with a pH of 7.4 at 37° C.

11. A block copolymer of the AB or ABA type with A being a polyester block and B being a polyether block.

12. A block copolymer according to claim 1 1, of the AB type and which is represented by formula I:
E-(O-D-CO—)ₙ—(O—CH₂—CH₂—)ₘ—O—F (formula I)
wherein
the structural unit E-(-O-D-CO—)ₙ— forms the block A,
—(O—CH₂—CH₂—)ₘ— forms the block B,
D may be, for each of the n units independently of one another:
—(CH(CH₃)—)ₓ or
—(CH₂—)ₓ or
—(CH₂—o—CH₂—CH₂—) or
—(CH₂—CH₂—CH₂—O—),
x is 1, 2, 3, 4 or 5,
E and F independently of one another denote H, methyl or ethyl and
n and m are statistically averaged and independently of one another denote an integer greater than 1.

13. A block copolymer according to claim 11, of the ABA type, and which is represented by formula TI:
E(-O-D-CO)ₙ—(O—CH₂—CH₂—)ₘ—O—(CO-D-O—)_{n′}-E (formula II),
wherein
the structural units E-(-O-D-CO—)ₙ— and E-(-O-D-CO—)_{n′}— form the blocks A,
—(O—CH₂—CH₂—)ₘ— forms the block B,
D may be, for each of the n or n′ units independently of one another:
—(CH(CH₃)—)ₓ or
—(CH₂—)ₓ or
—(CH₂—o—CH₂—CH₂—) or
—(CH₂—CH₂—CH₂—O—),
x is 1, 2, 3, 4 or 5,
E is H, methyl or ethyl and
n, n′ and m are statistically averaged and independently of one another denote an integer greater than 1.

14. Block copolymer according to claim 11, of the AB type and which is represented by formula I:
E-(O-D-CO—)ₙ—(O—CH₂—CH₂—)ₘ—O—F (formula I)
wherein
the structural unit E-(-O-D-CO—)ₙ— forms the block A,
—(O—CH₂—CH₂—)ₘ— forms the block B,
D may be, for each of the n units independently of one another:
—(CH(CH₃)—)ₓ or
—(CH₂—)ₓ or
—(CH₂—o—CH₂—CH₂—) or
—(CH₂—CH₂—CH₂—O—),
x is 1, 2, 3, 4 or 5,
E and F independently of one another denote H, methyl or ethyl and
n and m are statistically averaged and independently of one another denote an integer greater than 1
and the B block makes up a proportion of between 0.1 and 4 wt. %.

15. Block copolymer according to claim 11, of the ABA type and which is represented by the formula TI:
E(-O-D-CO)ₙ—(O—CH₂—CH₂—)ₘ—O—(CO-D-O—)_{n′}-E (formula II),
wherein
the structural units E-(-O-D-CO—)ₙ— and E-(-O-D-CO—)_{n′}— form the blocks A,
—(O—CH₂—CH₂—)ₘ— forms the block B,
D may be, for each of the n or n′ units independently of one another:
—(CH(CH₃)—)ₓ or
—(CH₂—)ₓ or
—(CH₂—O—CH₂—CH₂—) or
—(CH₂—CH₂—CH₂—O—),
x is 1, 2, 3, 4 or 5,
E is H, methyl or ethyl and
n, n′ and m are statistically averaged and independently of one another denote an integer greater than 1,
and the B block makes up a proportion of between 0.1 and 4 wt. %.

16. A block copolymer according to claim 11, wherein A is synthesised from a mixture of monomer components selected from the group consisting of a) D- and L-lactide, preferably in the ratio 1:1, b) L-lactide and glycolide, c) D,L-lactide and glycolide, d) L-lactide and epsilon-caprolactone, e) L-lactide and dioxanone, f) L-lactide and trimethylene carbonate.

17. A block copolymer according to claim 11, wherein the proportion by weight of the B block is between 1 and 3 wt. %.

18. A block copolymer according to claim 11, wherein the A block is synthesised from units of the L-lactide, D-lactide, meso-lactide or DL-lactide.

19. A block copolymer according to claim 18, wherein the A block is synthesised from units of the L-lactide.

20. A block copolymer according to claim 18, wherein the A block is synthesised from units of the DL-lactide.

21. A block copolymer of the AB type, represented by formula I according to claim 12, wherein the A block is synthesised from statistically distributed (randomised) units of the L-lactide and of the DL-lactide.

22. A block copolymer of the ABA type, represented by formula II according to claim 13, wherein the A block is synthesised from statistically distributed (randomised) units of the L-lactide and of the DL-lactide.

23. A block copolymer according to claim 21, wherein the molar proportion of L-lactide in the A block is between 60 and 90%.

24. A block copolymer according to claim 22, wherein the molar proportion of L-lactide in the A block is between 60 and 90%.

25. A block copolymer of the AB type, represented by formula I according to claim 12, wherein the A block is synthesised from statistically distributed (randomised) units of the L-lactide and of the DL-lactide with a molar proportion of the L-lactide of between 85 and 99%.

26. A block copolymer of the ABA type, represented by formula II according to claim 13, wherein the A block is synthesised from statistically distributed (randomised) units of the L-lactide and of the DL-lactide with a molar proportion of the L-lactide of between 85 and 99%.

27. A block copolymer according to claim 25, wherein the molar proportion of L-lactide in the A block is between 87 and 95%.

28. A block copolymer according to claim 26, wherein the molar proportion of L-lactide in the A block is between 87 and 95%.

29. A block copolymer of the AB type, represented by formula I according to claim 12, wherein the A block consists of statistically distributed units of the DL-lactide and of the glycolide and the inherent viscosity has a value of more than 0.8 dl/g.

30. A block copolymer of the ABA type, represented by formula II according to claim 13, wherein the A block consists of statistically distributed units of the DL-lactide and of the glycolide and the inherent viscosity has a value of more than 0.8 dl/g.

31. A block copolymer according to claim 29, wherein the molar proportion of DL-lactide in the A block is between 50 and 80%.

32. A block copolymer according to claim 30, wherein the molar proportion of DL-lactide in the A block is between 50 and 80%.

33. A block copolymer of the AB type, represented by formula I according to claim 12, wherein the A block consists of statistically distributed units of L-lactide, D-lactide, meso-lactide or DL-lactide and of epsilon-caprolactone.

34. A block copolymer of the ABA type, represented by formula II according to claim 13, wherein the A block consists of statistically distributed units of L-lactide, D-lactide, meso-lactide or DL-lactide and of epsilon-caprolactone.

35. A block copolymer according to claim 33, wherein the A block consists of statistically distributed units of L-lactide or DL-lactide and of epsilon-caprolactone.

36. A block copolymer according to claim 34, wherein the A block consists of statistically distributed units of L-lactide or DL-lactide and of epsilon-caprolactone.

37. A block copolymer of the AB type, represented by formula I according to claim 12, wherein the A block consists of statistically distributed units of L-lactide, D-lactide, meso-lactide or DL-lactide and of dioxanone.

38. A block copolymer of the ABA type, represented by formula II according to claim 13, wherein the A block consists of statistically distributed units of L-lactide, D-lactide, meso-lactide or DL-lactide and of dioxanone.

39. A block copolymer according to claim 37, wherein the A block consists of statistically distributed units of L-lactide or DL-lactide and of dioxanone.

40. A block copolymer according to claim 38, wherein the A block consists of statistically distributed units of L-lactide or DL-lactide and of dioxanone.

41. A block copolymer of the AB type, represented by formula I according to claim 12, wherein the A block consists of statistically distributed units of L-lactide, D-lactide, meso-lactide or DL-lactide and of trimethylene carbonate.

42. A block copolymer of the ABA type, represented by formula II according to claim 13, wherein the A block consists of statistically distributed units of L-lactide, D-lactide, meso-lactide or DL-lactide and of trimethylene carbonate.

43. A block copolymer according to claim 41, wherein the A block consists of statistically distributed units of L-lactide or DL-lactide and of trimethylene carbonate.

44. A block copolymer according to claim 42, wherein the A block consists of statistically distributed units of L-lactide or DL-lactide and of trimethylene carbonate.

45. A block copolymer of the AB type, represented by formula I according to claim 12, wherein the proportion by weight of the B block is between 0.01 and 25%.

46. A block copolymer of the ABA type, represented by formula I according to claim 13, wherein the proportion by weight of the B block is between 0.01 and 25%.

47. A block copolymer according to claim 45, wherein the proportion by weight of the B block is between 0.01 and 20%.

48. A block copolymer according to claim 46, wherein the proportion by weight of the B block is between 0.01 and 20%.

49. A block copolymer according to claim 45, wherein the proportion by weight of the B block is between 0.1 and 10%.

50. A block copolymer according to claim 46, wherein the proportion by weight of the B block is between 0.1 and 10%.

51. A block copolymer according to claim 45, wherein the proportion by weight of the B block is between 0.1 and 4%.

52. A block copolymer according to claim 46, wherein the proportion by weight of the B block is between 0.1 and 4%.

53. A block copolymer of the AB type, represented by formula I according to claim 12, wherein the numerically average block length of the B block is between 500 and 10000 Dalton.

54. A block copolymer of the ABA type, represented by formula II according to claim 13, wherein the numerically average block length of the B block is between 500 and 10000 Dalton.

55. A block copolymer according to claim 53, wherein the numerically average block length of the B block is between 1000 and 8000 Dalton.

56. A block copolymer according to claim 54, wherein the numerically average block length of the B block is between 1000 and 8000 Dalton.

57. A block copolymer of the AB type, represented by formula I according to claim 12, wherein the inherent viscosity is between 0.1 and 6 dl/g.

58. A block copolymer of the ABA type, represented by formula II according to claim 13, wherein the inherent viscosity is between 0.1 and 6 dl/g.

59. A block copolymer according to claim 57, wherein the inherent viscosity is between 0.5 and 5 dl/g.

60. A block copolymer according to claim 58, wherein the inherent viscosity is between 0.5 and 5 dl/g.

61. A block copolymer according to claim 57, wherein the inherent viscosity is between 0.6 and 3 dl/g.

62. A block copolymer according to claim 58, wherein the inherent viscosity is between 0.6 and 3 dl/g.

63. A block copolymer according to claim 57, wherein the inherent viscosity is between 0.7 and 2.75 dl/g.

64. A block copolymer according to claim 58, wherein the inherent viscosity is between 0.7 and 2.75 dl/g.

65. An implant made from a material which is a mixture of:
(a) a block copolymer of the AB or ABA type with A being a polyester block and B being a polyether block, and
(b) an absorbable polyester A with recurring units which are independently selected from the group consisting of L-lactide, D-lactide, DL-lactide or meso-lactide, glycolide, trimethylene carbonate, epsilon-caprolactone and dioxanone.
